(19) United States
(12) Patent Application Publication
(10) Pub. No.: US 2010/0221306 A1
(43) Pub. Date: Sep. 2, 2010

(54) ADHESIVE GEL SHEET FOR LIVING ORGANISMS AND SHEET FORM COSMETICS COMPRISING THE SAME

(75) Inventor: Shigetomo Tsujihata, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Minato-ku, Tokyo (JP)

(21) Appl. No.: 12/679,288

(22) PCT Filed: Aug. 21, 2008
(86) PCT No.: PCT/JP2008/065346

§ 371 Date: Mar. 19, 2010

(30) Foreign Application Priority Data
Sep. 20, 2007 (JP) 2007-244490

(51) Int. Cl. A61K 8/65; A61K 8/02; A61Q 19/00

(52) U.S. Cl. 424/443; 514/12

(57) ABSTRACT

To provide an adhesive gel sheet for living organisms which may allow efficient permeation of the skin by carotenoid, and also provide a sheet-form cosmetic using the same, It is provided an adhesive gel sheet for living organisms which includes a hydrogel having a carotenoid-containing O/W emulsion, gelatin or a derivative thereof and a hydrophilic polymer, and further comprises a polyvalent inorganic salt, wherein the content of the polyvalent inorganic salt is 0.1% or less by mass of the entire mass of the hydrogel. It is preferable that the hydrophilic polymer be a polysaccharide that forms a thermoreversible gel.

### BACKGROUND OF THE INVENTION

1. Technical Field

The present invention relates to an adhesive gel sheet for living organisms for use in the fields of drugs, quasi drugs, cosmetics, hygienic goods, sundries and the like, and to sheet-form cosmetics using the same.

2. Background Art

Adhesive gel sheets for living organisms are used for: packs and adhesive patches used in cosmetic treatment, facial treatment, skin treatment and the like; skin-permeable components and anti-inflammatory/analgesic components of active components of support materials; and adhesive tapes for living organisms, wound dressings and the like for the purpose of wound protection, drug fixation. These gel sheets are adhered to the skin, and enhance the physiological action of the skin by increasing the temperature and water content (water retentivity) of the skin. Thereby, movement of active ingredients in the sheet, through their permeation into the skin, is enhanced by the increased temperature and moisture of the skin.

It is known that gel sheets having the above functions contain collagen and polysaccharides such as chitin, chitosan, alginic acid and cellulose as constituents (for example, Japanese Patent Application Laid-Open (JP-A) No. 3-81213). However, it has been difficult to cause liposoluble active components to permeate into the skin by using such gel sheets.

At the same time, use of a fine carotenoid-containing O/W emulsion for improving the skin permeability of liposoluble components is disclosed in, for example, Japanese Patent Application Laid-Open (JP-A) 2005-75817 and the like. However, in the cream disclosed in JP-A 2005-75817, the skin permeability of carotenoid has been inadequate, and the effect has been insufficient in practice.

### SUMMARY OF THE INVENTION

The present invention has been made in view of the above circumstances and provides adhesive gel sheets for living organisms and sheet form cosmetics comprising the same.

A first aspect of the present invention provides an adhesive gel sheet for living organisms which comprises a hydrogel including a carotenoid-containing O/W emulsion, gelatin or a derivative thereof and a hydrophilic polymer; and further comprising a polyvalent inorganic salt, wherein the content of the polyvalent inorganic salt is 0.1% or less by mass of the entire mass of the hydrogel.

A second aspect of the present invention provides a sheet form cosmetic comprising the adhesive gel sheet for living organisms according to the first aspect.

### DETAILED DESCRIPTION OF THE INVENTION

### Disclosure of Invention

The present invention provides an adhesive gel sheet for living organisms which may make carotenoid permeate the skin efficiently and it also provides a sheet form cosmetic comprising the same. The adhesive gel sheet is an adhesive gel sheet for living organisms which comprises a hydrogel including a carotenoid-containing O/W emulsion, gelatin or a derivative thereof and a hydrophilic polymer; and further comprises a polyvalent inorganic salt, wherein the content of the polyvalent inorganic salt is 0.1% or less by mass of the entire mass of the hydrogel. It is preferable that the hydrophilic polymer be a polysaccharide that forms a thermoreversible gel.

### Problems to be Solved by the Invention

In view of the above-mentioned situation, the object of the invention is to provide an adhesive gel sheet for living organisms which may make carotenoid permeate the skin efficiently and a sheet form cosmetic comprising the same.

### Means for Solving the Problems

The adhesive gel sheet for living organisms of the invention comprises a hydrogel including a carotenoid-containing O/W emulsion, gelatin or a derivative thereof and a hydrophilic polymer; and further comprises a polyvalent inorganic salt, wherein the content of the polyvalent inorganic salt is 0.1% or less by mass.

It is preferable that the hydrophilic polymer be a polysaccharide that forms a thermoreversible gel. It is preferable that the polysaccharide be at least one species selected from the group consisting of agar, glucomannan, guar gum, locust bean gum, carrageenan, gellan gum, native gellan gum and xanthan gum.

It is preferable that the adhesive gel sheet for living organisms of the invention further comprise a polyhydric alcohol compound. The volume average particle diameter of the O/W emulsion is preferably from 10 nm or more to 200 nm or less. It is preferable that the carotenoid be astaxanthin and/or an ester thereof.

The sheet form cosmetic of the invention is a product produced using an adhesive gel sheet for living organisms.

In the invention, the “adhesive gel sheet for living organisms” includes packs, and adhesive patches used in cosmetic treatment, facial treatment, skin treatment and the like; skin-permeable components and anti-inflammatory/analgesic components of active components of support materials; and adhesive tapes for living organisms, wound dressing agents and the like for the purpose of wound protection, drug fixation. It is an adhesive sheet to be used while being stuck directly on the skin for the purpose of retention and permeation to the skin of an active ingredient or moisture. The adhesive gel sheet for living organisms is useful as a cosmetic such as a pack for giving moisture or an active ingredient to the skin while being stuck on the skin.

### BEST MODE FOR CARRYING OUT THE INVENTION

The invention is described in detail below.

### (Adhesive Gel Sheet for Living Organisms)

The adhesive gel sheet for living organisms of the invention comprises a hydrogel including a carotenoid-containing O/W emulsion, gelatin or a derivative thereof and a hydrophilic polymer; and further comprises a polyvalent inorganic salt, wherein the content of a polyvalent inorganic salt is 0.1% or less by mass of the entire mass of the hydrogel.

In the invention, the content of a polyvalent inorganic salt, which may serve as a crosslinking agent for gelatin, is adjusted to 0.1% or less by mass of the entire mass of the hydrogel. Thereby, the hardening of the hydrogel is inhibited and, when the gel sheet is stuck, the hydrogel is softened, so that the carotenoid-containing O/W emulsion is rendered easy to ooze out from the hydrogel.

<Hydrogel>

### [Gelatin]

The adhesive gel sheet for living organisms of the invention comprises gelatin or a derivative thereof in hydrogel. The carotenoid-containing O/W emulsion is stabilized due to the protective colloid function of the gelatin and, in addition, when the adhesive gel sheet for living organisms is stuck on a skin, it may improve the skin permeability due to the melting of the gelatin and the closing effect of the hydrogel.

Gelatin is a hydrolyzed protein of collagen. The method for the preparation thereof is not restricted and products produced by acid treatment or alkali treatment using bovine-bone, oxhide, pigskin, fish scales, and the like as a raw material are common. Products prepared by an enzymic process may also be used.

The jelly strength of the gelatin used in the invention, as measured in accordance with JIS K6503, is preferably from 50 to 300 g, and it is more preferably from 100 to 280 g. By forming a hydrogel using a gelatin has a jelly strength within such ranges, the stability and the skin permeability of a carotenoid-containing emulsion are improved.

As a derivative of gelatin, conventional derivatives may be used. Examples thereof include acid anhydride-adducts of gelatin (e.g., phthalated gelatin, succinated gelatin and trimellitated gelatin), lactone-adducts of gelatin (e.g., glucono-δ-lactone-added gelatin), acylated gelatins (e.g., acetylated gelatin), etherified gelatins (e.g., methyl-esterified gelatin), and gelatin-organic acid salts (gelatin-acetatic acid salt, gelatin-stearic acid salt, gelatin-benzoic acid salt).

Among these, pig skin-derived gelatin, fish-derived gelatin, succinated gelatin, phthalated gelatin, and trimellitated gelatin are preferred as the gelatin or a derivative thereof to be used for the adhesive gel sheet for living organisms of the invention from the viewpoint of the affinity with living organisms and release of active ingredients.

Regarding the above-mentioned gelatins or derivatives thereof, a single kind of gelatin may be used or, alternatively, two or more kinds of gelatins may be used in combination. A single kind of gelating derivative may be used or, alternatively, two or more kinds of gelatin derivative may be used in combination. Gelatin and a derivative of gelatin may also be used in combination. It is also permissible to use collagen together.

The weight average molecular weight, as measured by gel permeation chromatography (GPC), of gelatin or a derivative thereof in the invention is preferably from 5,000 to 1,000,000, more preferably from 5,000 to 300,000, and particularly preferably from 10,000 to 300,000.

The content of the gelatin or a derivative thereof in the hydrogel in the invention is preferably from 0.05 to 20% by mass, more preferably from 0.1 to 10% by mass, and particularly preferably from 0.2 to 5% by mass. If the content is 0.05% or more by mass, the skin permeability of the active components in the hydrogel is good and if it is 20% or less by mass, the handling property is good.

[Hydrophilic Polymer]

As the hydrophilic polymer to be used in the invention, any of synthetic polymers or natural polymers having a hydrophilic functional group (e.g. a hydroxyl group, a carboxyl group, a sulfo group, a phospho group, carbamoyl, an amino group, an ammonio group, and an ethylene oxy group) may be used. These may be used alone or alternatively may be used as a mixture of two or more of them.

Examples of synthetic polymers having a hydrophilic group which are suitable for the invention include vinyl alcohol (co)polymer, 2-hydroxyethyl acrylate (co)polymer, acrylic acid (co)polymer, methacrylic acid (co)polymer, maleic acid (co)polymer, itaconic acid (co)polymer, p-vinylbenzoic acid (co)polymer, 2-acrylamido-2-methyl-1-propanesulfonic acid (co)polymer, styrene sulfonic acid (co)polymer, acrylamide(co)polymer, acryloylmorpholine (co)polymer, N-vinylpyrrolidone (co)polymer, vinylamine(co)polymer, N,N-dimethyldiallylammonium chloride (co)polymer, 2-methacryloyloxyethylammonium chloride (co)polymer, polyethylene glycol methacrylate (co)polymer, and polyethyleneimine.

Examples of natural polymers having a hydrophilc group include neutral polysaccharides (cellulose, amylose, amylopectin, dextran, pullulan, inulin, galactan, mannan, xylan, arabinan, glucomannan, galactomannan, hydroxyethylcellulose, methylcellulose, and the like), anionic polysaccharides (pectic acid, alginic acid, agarose, agar, carrageenan, fucoidan, hyaluronic acid, chondroitin sulfate, heparin, gellan gum, native gellan gum, xanthan gum, carboxymethylcellulose, and the like), cationic polysaccharides (chitin, chitosan, cationized cellulose, and the like), and proteins (casein, elastin, polypeptide).

Among these, hydrophilic polymers which are highly active in increasing the viscosity and gelating are preferable as the hydrophilic polymer used for the invention. One or more kinds of apolysaccharides that form thermoreversible gel are particularly preferable.

Polysaccharides that form thermoreversible gel may be either polysaccharides which are each able to form alone thermoreversible gel or polysaccharides which, in a combination of two or more of them, are able to form thermoreversible gel. Specific examples include agar, glucomannan, guar gum, locust bean gum, carrageenan, gellan gum, native gellan gum, xanthan gum, xyloglucan, and agarose. Among these, combinations of agar, gellan gum, native gellan gum and xanthan gum with glucomannan or locust bean gum, combinations of κ-carrageenan with glucomannan or locust bean gum, and a combination of gellan gum with glucomannan are particularly preferred.

From the viewpoint of stabilization of the structure of a hydrogel at normal temperature and improvement in handleability thereof, the above-mentioned hydrophilic polymers preferably have a weight average molecular weight of from 10,000 to 5,000,000, and more preferably from 50,000 to 2,000,000.

The content of the hydrophilic polymer in the hydrogel in the invention is preferably from 0.05 to 10% by mass, more preferably from 0.1 to 5% by mass, and particularly preferably from 0.2 to 2% by mass. If the content is 0.05% or more by mass, the hydrogel is easy to handle. If the content is 10% or less by mass, the carotenoid-containing emulsion is good in skin permeability.

[Carotenoid-Containing O/W Emulsion]

The adhesive gel sheet for living organisms of the invention comprises a carotenoid-containing O/W emulsion in the hydrogel in order to increase, for example, the skin-beautifying effect by skin permeation. Examples of the carotenoids include actinioerythrol, astaxanthin, bixin, kantaxanthin, capxanthin, capsorbin, β-8′-apo-cartenal, β-12′-apo-cartenal, α-carotene, β-carotene, γ-carotene, β-cryptoxanthin, lutein, lycopene, violeritorin, zeaxanthin, fucoxanthin and their derivatives.

Among these, astaxanthin, lutein, zeaxanthin and β-cryptoxanthin are preferred as carotenoid in the invention. In particular, astaxanthin, whose antioxidant effect, anti-inflammatory effect, anti-skin-aging effect and whitening effect have been recognized, is preferred.

The carotenoid-containing O/W emulsion may comprise, in amounts generally used, other ingredients, such as emulsifiers, which may generally be comprised in phases of emulsion compositions. Such additional ingredients include ingredients such as polyhydric alcohols disclosed in this description.

The volume average particle diameter of the O/W emulsion used in the invention is preferably from 10 to 200 nm, and particularly preferably from 10 to 100 nm.

The volume mean diameter of the emulsion particle of the invention can be measured with the commercially available particle size distribution measuring device. Optical microscopy, confocal laser microscopy, electron microscopy, atomic force microscopy, static light scattering method, laser diffraction method, dynamic light scattering method, centrifugal precipitation method, electric pulse measurement method, chromatography method, ultrasonic damping method and the like are known as the particle size distribution measurement method of an emulsion, and devices corresponding to the respective principle are commercially available.

From the range of the volume mean diameter of the emulsion particle in the invention and easy measurement, dynamic light scattering method is preferred in the emulsion particle size measurement of the invention. The commercially available measurement devices using dynamic light scattering include Nanotrac UPA (Nikkiso Co., Ltd.), dynamic light scattering particle size distribution measuring device LB-550 (Horiba, Ltd.) and fiber-optics particle size analyzer FPAR-1000 (Otsuka Electronics Co., Ltd.).

In the invention, a value measured with a fiber-optics particle size analyzer FPAR-1000 (manufactured by Otsuka Electronics Co., Ltd.) at 25° C. is adopted as the volume average particle diameter of a carotenoid-containing O/W emulsion.

The method for measuring the volume average particle diameter is practiced with a glass tube for measurement after such dilution with pure water that the concentration of an oily phase ingredient might become within the range of from 0.1 to 1% by mass. The volume average particle diameter may be determined as an accumulative (50%) value in a measurement under a dispersion medium refractive index of 1.3313 (pure water) and a dispersion medium viscosity of 0.8846 cp (pure water).

The method for preparing a carotenoid-containing O/W emulsion used in the invention is not particularly restricted. For example, the method disclosed in JP-A No. 2005-75817 may be used. Alternatively, it is preferable that the method comprises a step of a) dissolving a water-soluble emulsifier in an aqueous medium to obtain an aqueous phase composition, b) mixing and dissolving carotenoid, tocopherol, lecithin and, if necessary, other oils and fats to obtain an oily phase composition (oily phase ingredient), and c) mixing the aqueous phase composition and the oily phase composition under stirring to perform emulsification dispersion, obtaining an emulsion composition.

In the emulsification dispersion, it is particularly preferable to use two or more kinds of emulsifying apparatus together by a method such as that in which emulsification is performed by use of a normal emulsifying apparatus utilizing shearing action such as a stirrer, an impeller, a homomixer and a continuous flow-type shearing apparatus, and then performing pass through a high-pressure homogenizer. By use of a high-pressure homogenizer, it is possible to make an emulsified matter into more uniform fine particle droplets.

The content of the carotenoid in the hydrogel of the invention is preferably from 0.0001 to 0.5% by mass, more preferably from 0.0005 to 0.1% by mass, and particularly preferably from 0.001 to 0.05% by mass. If the carotenoid content is 0.0001% or more by mass, an effect (skin-beautifying effect, and the like) is felt after the adhesive gel sheet for living organisms of the invention is stuck on the skin. If the content is 0.5% or less by mass, the coloring to the skin may be suppressed and uncomfortable feeling is hardly produced.

The content of the carotenoid-containing emulsion in the hydrogel of the invention is preferably from 0.01 to 5% by mass, and more preferably from 0.05 to 1% by mass from the viewpoint of stability and skin permeability of fine particles.

[Polyvalent inorganic salt]

In the invention, the content of the polyvalent inorganic salt is 0.1% or less by mass of the mass of the hydrogel. The polyvalent inorganic salt is a substance which may serve as a cross-linking agent for gelatin or a hydrophilic polymer. By adjusting the amount of the polyvalent inorganic salt to a very small amount as small as 0.1% or less by mass of the hydrogel, it is possible to inhibit the hardening of the hydrogel. As a result, the active ingredients or the like in the hydrogel become prone to ooze out of the hydrogel, and it is possible to increase the skin permeability of the active ingredients in the hydrogel when the adhesive gel sheet for living organisms of the invention is stuck to the skin or the like.

If the content of the polyvalent inorganic salt is greater than 0.1% by mass of the mass of the hydrogel, the skin permeability of the carotenoid-containing emulsion decreases.

The content of the polyvalent inorganic salt is preferably 0.05% or less by mass, and more preferably 0.01% or less by mass of the mass of the hydrogel. The most preferable embodiment is that the content is 0% by mass, namely, that no polyvalent inorganic salt is comprised in the hydrogel.

Examples of the polyvalent inorganic salts include water-soluble salts, such as calcium chloride, magnesium chloride, aluminum chloride, potash alum, ammonium alum, iron alum, aluminum sulfate, ferric sulfate, magnesium sulfate, and aluminum polychloride; and salts which are insoluble or slightly soluble in water, such as calcium hydroxide, ferric hydroxide, aluminum hydroxide, calcium phosphate, barium sulfate, barium hydroxide, aluminum allantoinate, and bismuth subnitrate.

[Other Ingredients]

It is preferable that the adhesive gel sheet for living organisms of the invention further comprise a polyhydric alcohol compound in the hydrogel in order to demonstrate various effects. It may further comprise an excipient, an additive, and the like according to necessity.

—Polyhydric Alcohol—

It is preferable, from the viewpoint of skin permeability in use of the hydrogel itself as an adhesive gel sheet for living organisms, that the hydrogel used in the invention further comprise a polyhydric alcohol compound.

Examples of polyhydric alcohols include glycerols (glycerol, diglycerine, and the like), glycols (diethylene glycol, triethylene glycol, tetraethylene glycol, polyethylene glycol, 1,2-propanediol, 1,3-propanediol, dipropylene glycol, 1,2-butanediol, 1,3-butanediol, 1,2-pentanediol, 1,2-hexanediol, 1,2-octanediol, and the like), sugars (glucose, fructose, mannose, galactose, xylose, arabinose, glucosamine, N-acetylglucosamine, sucrose, lactose, maltose, isomaltose, trehalose, cellobiose, kojibiose, sophorose, maltotriose, raffinose, stachyose, and the like), and sugar alcohols (glycerol, threitol, erythritol, arabinitol, xylitol, ribitol, mannitol, sorbitol, galactitol, inositol, and the like). These polyhydric alcohols may be used solely or as a mixture of two or more them.

Among them, compounds having a 1,2- or 1,3-diol structure are preferred for the purpose of improving the skin permeability of the adhesive gel sheet for living organisms of the invention. For example, glycerol, 1,2-propanediol, 1,2-butanediol, 1,3-butanediol, 1,2-pentanediol, and 1,2-hexanediol are more preferred. Glycerol, 1,2-propanediol, and 1,3-butanediol are particularly preferred.

The content of a polyhydric alcohol in the hydrogel in the invention is preferably 50% or less by mass, and particularly preferably from 1 to 20% by mass.

If the content of a polyhydric alcohol in the hydrogel in the invention is 50% or less by mass, it is possible to prevent the gel strength from decreasing.

—Excipient —

In the adhesive gel sheet for living organisms of the invention, an excipient may add to the hydrogel for improvement in the stability in form of the adhesive gel sheet for living organisms. As the excipient, organic or inorganic fine particles may be preferably used. As organic particles, polystyrene particles, polymethacrylate particles, and microcrystalline cellulose, which are known in the art, are preferred. As inorganic particles, silica, alumina, calcium carbonate, kaolin, clay mineral, and the like are preferred. Among these, silica or clay mineral is preferred. In particular, gaseous phase method silica and synthetic smectite having an average particle diameter of 200 nm or less are particularly preferable.

The content of an excipient in the hydrogel in the invention is preferably 10% or less by mass, and particularly preferably from 1 to 5% by mass.

—Additives —

Depending upon the intended use of the adhesive gel sheet for living organisms of the invention, various active ingredients and additives may be blended. Examples of such active ingredients or additives include humectants, thickeners, flavoring agents, coloring agents, stabilizers, antioxidants, ultraviolet absorbers, tackifiers, pH adjusters, chelating agents, surfactants, antiseptic agents and antibacterial agents in addition to medicinal ingredients for the purposes of cosmetic and facial treatment and skin treatment.

Examples of the humectants include amino acids, α-hydroxy acid salts (e.g. sodium lactate, potassium lactate and potassium gluconate), urea, sodium pyrrolidone carboxylate, betaine and whey. These may be used solely or as a mixture of two or more of them.

The content of a humectant in the hydrogel is preferably from 0.1 to 10% by mass, and more preferably from 0.5 to 5% by mass.

As the medicinal ingredients, those other than the aforementioned carotenoid may be used properly. As such medicinal ingredients, any substances are not particularly restricted as long as they have heretofore been used, for example, in the fields of drugs, quasi drugs, cosmetics, hygienic goods and sundries. Examples thereof include natural components such as angelica keiskei extract, persea gratissima (avocado) fruit extract, hydrangea serrata leaf extract, althea extract, arnica extract, aloe extract, prunus armeniaca (apricot) kernel extract, prunus armeniaca (apricot) kernel extract, ginkgo biloba extract, foeniculum vulgare (fennel) extract, turmeric extract, oolong tea extract, rose fruit extract, echinacea angustifolia leaf exract, scutellaria baicalensis root extract, phellodendron bark extract, coptis japonica root extract, hordeum vulgare seed extract, hypericum perforatum extract, lamium album extract, nasturtium officinale extract, citrus aurantium dulcis (orange) fruit extract, dehydrated seawater, seaweed extract, hydrolyzed elastin, hydrolyzed wheat powder, hydrolyzed silk, chamomilla recutita (matricaria) extract, daucus carota sativa (carrot) root extract, artemisia capillaris flower extract, glycyrrhiza extract, hibiscus tea extract, pyracantha fortuneana fruit extract, actinidia chinensis (kiwi) fruit extract, cinchona extract, cucumis sativus (cucumber) fruit extract, guanosine, gardenia florida extract, sasa veitchii extract, sophora angustifolia extract, walnut shell extract, citrus grandis (grapefruit) fruit extract, clematis vitalba leaf extract, chlorella vulgaris extaract, morus alba leaf extract, gentiana lutea extract, black tea extract, yeast extract, arctium lappa root extract, fermented rice bran extract, rice germ oil, symphytum officinale leaf extract, cowberry extract, asiasarum root extract, family of bupleurum extract, umbilical cord extract, saponaria officinalis extract, bamboo extract, crataegus cuneata fruit extract, zanthoxylum fruit extract, corthellus shiitake (mushroom) extract, rehmania root extract, lithospermum erythrorhizone root extract, perilla herb extract, linden extract, spiraea ulmaria extract,

peony root extract, acorns calamus root extract, birch extract, equisetum arvense extract, hedera helix (ivy) extract, quickthorn extract, bourtree extract, nosebleed extract, mentha piperita (peppermint) leaf extract, salvia officinalis (sage) leaf extract, malva sylvestris (mallow) extract, cnidium officinale root extract, swertia japonica extract, glycine soja (soybean) seed extract, jujube fruit extract, thymus vulgaris (thyme) extract, green tea extract, eugenia caryophyllus (clove) flower extract, Imperata cylindrical extract, citrus unshiu peel extract, japanese angelica root extract, calendula officinalis flower extract, peach kernel extract, bitter orange peel extract, houttuynia cordata extract, solanum lycopersicum (tomato) extract, fermented soybeans extract, panax ginseng root extract, allium sativum (garlic) extract, wild rose extract, hibiscus extract, ophiopogonis tuber extract, carum petroselinum (parsley) extract, honey, hamamelis virginiana (witch hazel) extract, parietaria officinalis extract, isodonis japonicus extract, bisabolol, eriobotrya japonica leaf extract, coltsfoot extract, butterbur sprout extract, poria cocos extract, ruscus aculeatus root extract, vitis vinifera (grape) fruit extract, propolis, luffa cylindrica fruit extract, carthamus tinctorius (safflower) flower extract, peppermint extract, tilia platyphyllos flower extract, paeonia suffruticosa root extract, humulus lupulus (hops) extract, pinus sylvestris cone extract, aesculus hippocastanum (horse chestnut) extract, lysichiton camtschatcense extract, sapindus mukurossi peel extract, melissa officinalis (balm mint) leaf extract, prunus persica (peach) leaf extract, centaurea cyanus flower extract, eucalyptus leaf extract, saxifraga sarmentosa extract, yuzu extract, mugwort extract, lavandula angustifolia (lavender) extract, pyrus malus (apple) fruit extract, lactuca scariola sativa (lettuce) extract, citrus medica limonum (lemon) extract, astragalus sinicus extract, rosa centifolia flower extract, rosamarinus officinalis (rosemary) leaf extract, anthemis nobilis flower extract, royal jelly extract, plant-derived components and seaweed-derived components.

Further examples are oil ingredients such as sphingolipid, ceramide, cholesterol, cholesterol derivatives and phospholipid; anti-inflammatory agents such as ε-aminocaproic acid, glycyrrhizinic acid, β-glycyrrhizinic acid, lysozyme chloride, guaiazulene and hydrocortisone; vitamins such as vitamins A, B2, B6, C, D and E, calcium pantothenate, biotin, nicotinamide and vitamin C esters; active ingredients such as allantoin, diisopropylamine dichloroacetate and 4-aminomethyl cyclohexanecarboxylic acid; antioxidants such as CoQ10, flavonoid, tannin, lignan and saponin; cell activators such as α-hydroxy acid and β-hydroxy acid; blood circulation accelerators such as γ-orizanol and vitamin E derivatives; wound healing agents such as retinol and retinol derivatives; whitening agents such as arbutin, kojic acid, placenta protein, sulfur, ellagic acid, linolic acid, tranexamic acid and glutathione;

cepharanthin, glycyrrhiza extract, capsicum tincture, hinokitiol, iodinated garlic extract, pyridoxine hydrochloride, dl-α-tocopherol, dl-α-tocopherol acetate, nicotinic acid, nicotinic acid derivatives, calcium pantothenate, D-pantothenyl alcohol, acetyl pantothenyl ethyl ether, biotin, allantoin, isopropyl methyl phenol, estradiol, ethinyl estradiol, capronium chloride, benzalkonium chloride, diphenhydramine hydrochloride, takanal, camphor, salicylic acid, vanillylamide nonylate, vanillylamide nonanoate, piroctone olamine, glyceryl pentadecanoate, 1-menthol, mononitro guaiacol, resorcin, γ-aminobutyric acid, benzethonium chloride, mexiletine hydrochloride, auxin, estrogen, cantharis tincture, ciclosporin, hydrocortisone, polyoxyethylene sorbitan monostearate, and mentha oil.

If it is possible to administer to a living organism by permeation through the skin, it is also possible to add medicinal ingredients, such as analgesics, tranquilizers, antihypertensives, antibiotics, antihistaminics and antibacterial substances.

Active ingredients such as those medicinal ingredients may be added, in the form of emulsion, into a hydrogel, or alternatively, they may be added to a hydrogel directly.

The content of the active ingredient is not defined specifically because the effective amount of the ingredient varies depending on the material of the medicinal ingredients. In general, the content is preferably 0.001 to 10% by mass, and more preferably 0.05 to 5% by mass to the total amount of the hydrogel.

[Structure of Adhesive Gel Sheet for Living Organisms]

The adhesive gel sheet for living organisms of the invention may be constituted solely of a layer of a hydrogel like that described above, or alternatively, it may be of a multilayer structure further having other layers. It is also acceptable that two or more layers of a hydrogel are provided. When there are two or more hydrogel layers, the ingredients previously mentioned may be comprised in the same hydrogel layer, or alternatively, they may be comprised dividedly in two or more layers. When an adhesive gel sheet for living organisms forms a multilayer structure, each of the ingredients of the adhesive gel sheet for living organisms may be comprised in an amount within the aforementioned range in the adhesive gel sheet for living organisms as a whole.

Examples of the layers other than the hydrogel layer include a support layer and a protective sheet. From the viewpoint of improvement in the shape stability and the handleability of the adhesive gel sheet for living organisms of the invention, it is preferable to provide a support layer. It is preferable, from the viewpoint of protection of a surface of the hydrogel layer before use, to provide a protective layer.

It is preferable to use, as a support layer, sheet form supporting substrates known in the art such as non-woven fabric, woven fabric and plastic film, crosslinked gel (gelatin/glutaraldehyde crosslinked gel, polyacrylic acid/polyvalent metal ion crosslinked gel, and the like), physical gels (agarose gel, κ-carrageenan gel, and the like), water-insoluble films formed from a hydrophilic polymer (chitosan film, cellophane, κ-carrageenan cast film, and the like), and the like.

Among these, use of a transparent film having a thickness of 100 μm or less is generally preferable in the adhesive gel sheet for living organisms of the invention from the viewpoint of the shape stability and the handleability of the adhesive gel sheet for living organisms. In particular, water-insoluble films formed from a hydrophilic polymer, such as chitosan film, cellophane, and κ-carrageenan cast film, are preferred.

As a protection sheet, it is preferable to use a polyethylene film, a polypropylene film, a PET film, and the like. In particular, use of a polyethylene film having a thickness of 500 μm or less is preferred.

The thickness of an adhesive gel sheet for living organisms taken when the adhesive gel sheet for living organisms is constituted only of a hydrogel layer is preferably from 0.2 to 3 mm in order to demonstrate the effect of the skin permeability of a carotenoid-containing emulsion. Further taking into consideration the improvement in sticking stability, it is more preferably from 0.3 to 2 mm, and particularly preferably from 0.5 to 1.5 mm. If the thickness of an adhesive gel sheet for living organisms is 0.2 mm or more, its hydrogel is prevented from drying and the skin permeability is not affected when the adhesive gel sheet for living organisms is stuck on the skin. If the thickness of an adhesive gel sheet for living organisms is 3 mm or less, an emulsion oozes out readily from its hydrogel.

While the thickness of a support layer and that of a protective sheet are as mentioned supra, it is more preferable that the thickness of the support layer be from 5 to 80 μm and that the thickness of the protective sheet be from 50 to 500 μm in the case of a multilayer-structured adhesive gel sheet for living organisms which is constituted of a hydrogel layer and a support layer and/or a protective sheet. In such a case of multilayer structure, the thickness the hydrogel may be adjusted to be equal to the thickness of the hydrogel mentioned previously, or alternatively, it may be adjusted appropriately so that the overall thickness of the adhesive gel sheet for living organisms might become within the range of 0.4 to 3 mm.

[Method for Producing Adhesive Gel Sheet for Living Organisms]

The adhesive gel sheet for living organisms of the invention may be produced in accordance with a method generally used.

For example, when an adhesive gel sheet for living organisms is constituted only of a hydrogel, it may be produced in accordance with an ordinary method for producing the hydrogel. Specifically, a composition of a hydrogel including the aforementioned ingredients (a hydrogel composition) is heated and mixed to yield an aqueous solution, and then it is applied into a sheet-like form using an applicator such as a doctor blade. Then, the sheet-like hydrogel composition is cooled to complete its gelation. Thereby, an adhesive gel sheet for living organisms is obtained.

Addition of a carotenoid-containing emulsion to a hydrogel composition may be conducted either before the hydrogel composition gel is gelated (in a sol state) or after it is gelated.

In the case of an adhesive gel sheet for living organisms which has a support layer, it may be obtained easily by, for example, applying a mixed aqueous solution of the ingredients of a hydrogel as previously mentioned onto a sheet-like form supporting substrate to laminate a hydrogel layer.

In the case of further providing a protective sheet, the protective sheet may be laminated on the support layer-free surface of the hydrogel layer.

(Physical Properties of Adhesive Gel Sheet for Living Organisms)

It is preferable that the adhesive gel sheet for living organisms of the invention be highly transparent from the viewpoint of reducing the odd feeling in appearance when it is stuck to the skin.

The transparency in the invention may be evaluated using, as a measure, a value evaluated on the basis of a transmittance measured at a wavelength of 600 nm with a spectrophotometer. Regarding the transparency, it is preferable that the transmittance to distilled water measured by the aforementioned evaluation method be 60% or more by mass, and more preferably 98% or more by mass. If the transmittance is 60% or more by mass, it becomes easy to check the state of the skin in sticking. However, the adhesive gel sheet is not necessarily required to be transparent when it is applied to a use site where the appearance is not considered.

While the adhesive gel sheet for living organisms of the invention is not particularly limited in shape, it may be in a tape-like form and be supplied in a rolled form or it may be separate sheets which are independent from each other.

In the case of separate sheets, they may be in any shape. Examples of the shape include oval, circle, heart form, semicircle, semioval, square, rectangle, trapezoid, triangle, and combinations thereof. A shape in conformity with the application site, or a shape in which they may be stuck to use sites most properly may be designed appropriately. Further, the handleability of an adhesive gel sheet for living organisms may be improved by forming a convex portion or a concave portion for alignment in the center or the periphery of the adhesive gel sheet, or alternatively, by forming a cut or a cut-off portion depending on the configuration of the use part.

Such adhesive gel sheets for living organisms may be hermetically sealed one by one in packaging materials made of a sheet of air-impermeable sheet in order to prevent moisture or active ingredients from decreasing with time.

Organism parts to which the adhesive gel sheet for living organisms of the invention is applied may be the face (lips, cheeks, eye regions, regions above and beneath eyes, a nose, a forehead, an entire face), arms, legs, the chest, the abdomen, the back, the neck, and the like.

The adhesive gel sheet for living organisms may be adjusted not only in its shape previously mentioned but also in its area, thickness, adhesion property on the outermost surface of the hydrogel layer, and the like suitably depending on the organism part to which the sheet is applied.

For example, In a case of an application of the adhesive gel sheet for living organisms whose application site is the entire face, it is preferable that the sheet be formed into a shape in which the portions corresponding to the positions of the eyes and mouth have been removed and the portion corresponding to the position of the nose has been cut and that adjustment such as increasing the adhesion ability of the adhesive layer or reducing its thickness a little is conducted because the application area is large. It is also permissible to divide the shape for the face into two sections, namely, an upper section which is to be applied to the forehead and the portions around the eyes and nose and a lower section which is to be applied to the portion from around the mouth to the chin.

(Sheet Form Cosmetic)

The sheet form cosmetic of the invention may be constituted with an adhesive gel sheet for living organisms of the invention.

Because the adhesive gel sheet for living organisms of the invention may cause carotenoid to permeate the skin efficiently, the sheet form cosmetic of the invention is useful as, especially, a sheet form cosmetic like those mentioned above which is stuck on the face to give moisture or medicinal ingredients to the skin.

The disclosure of Japanese Patent Application No. 2007-244490 filed Sep. 20, 2007, is incorporated herein by reference in its entirety.

### EXAMPLES

Hereunder is a specific description of exemplary embodiments of the present invention with reference to Examples. However, the present invention is not limited to these Examples. In the examples, the word “part(s)” and the symbol “%” mean “part(s) by mass” and “% by mass”, respectively, unless otherwise specified.

### Example 1

### Preparation of Carotenoid-Containing Emulsion

An aqueous phase composition was obtained by heating the following ingredients at 70° C. to dissolve them in one hour.

| | |
|---|---|
| Sucrose oleate | 15 g |
| Decaglyceryl monooleate | 23 g |
| Glycerol | 500 g |
| Pure water | 322 g |

An oily phase composition was obtained by heating the following ingredients at 70° C. to dissolve them in one hour.

| | |
|---|---|
| Haematococcus algae extract (astaxanthin content = 20% by mass) | 40 g |
| Mixed tocopherols | 10 g |
| Lecithin (derived from soybean) | 90 g |

An emulsion was obtained by stirring the aqueous phase composition with a homogenizer (at 10,000 rpm) while keeping it at 70° C., and then adding the oily phase composition thereto. The resulting emulsion was subjected to high-pressure emulsification under a pressure of 200 MPa using an Ultimizer HJP-25005 (manufactured by Sugino Machine Ltd.). The volume average particle diameter of the resulting emulsion was measured, with a fiber-optics particle size analyzer FPAR-1000 (manufactured by Otsuka Electronics Co., Ltd.) at 25° C., to be 90 nm.

<Preparation of Hydrogel>

A sol-like material was obtained by heating and kneading the following components at 80° C.

| | |
|---|---|
| κ-Carrageenan | 0.20 g |
| Glucomannan (from konnyaku potato) | 0.20 g |
| Pigskin-derived acid-treated gelatin | 0.50 g |
| 1,3-Butanediol | 5.0 g |
| Pure water | 44.09 g |

This sol-like material was cooled to 60° C., and then 0.1 g of the carotenoid-containing emulsions was added thereto, followed by stirring uniformly. The resultant was spread with a doctor blade so that it might come to have a thickness of 1 mm, and then it was left at rest at 25° C. for 24 hours. Thereby, an adhesive gel sheet for living organisms of Example 1 was obtained.

### Example 2

An adhesive gel sheet for living organisms of Example 2 was obtained in the same manner as Example 1 except for changing the composition of the hydrogel in the production process of the adhesive gel sheet for living organisms of Example 1 to the composition shown below.

| | |
|---|---|
| κ-Carrageenan | 0.20 g |
| Glucomannan (from konnyaku potato) | 0.20 g |
| Pigskin-derived acid-treated gelatin | 0.50 g |
| Magnesium chloride | 0.05 g |
| 1,3-Butanediol | 5.0 g |
| Pure water | 44.04 g |

### Example 3

An adhesive gel sheet for living organisms of Example 3 was obtained in the same manner as Example 1 except for changing the composition of the hydrogel in the production process of the adhesive gel sheet for living organisms of Example 1 to the composition shown below.

| | |
|---|---|
| Gellan gum | 0.20 g |
| Native gellan gum | 0.20 g |
| Pigskin-derived acid-treated gelatin | 0.50 g |
| 1,3-Butanediol | 5.0 g |
| Pure water | 44.09 g |

### Example 4

An adhesive gel sheet for living organisms of Example 4 was obtained in the same manner as Example 1 except for changing the composition of the hydrogel in the production process of the adhesive gel sheet for living organisms of Example 1 to the composition shown below.

| | |
|---|---|
| Xanthan gum | 0.30 g |
| Locust bean gum | 0.30 g |
| Pig skin-derived acid-treated gelatin | 0.40 g |
| Succinated gelatin | 0.1 g |
| 1,3-Butanediol | 5.0 g |
| Pure water | 43.89 g |

### Example 5

An adhesive gel sheet for living organisms of Example 5 was obtained in the same manner as Example 1 except for changing the composition of the hydrogel in the production process of the adhesive gel sheet for living organisms of Example 1 to the composition shown below.

| | |
|---|---|
| Poly(sodium acrylate) | 1.00 g |
| Polyacrylic acid | 1.00 g |
| Pigskin-derived acid-treated gelatin | 0.40 g |
| Calcium chloride | 0.05 g |
| 1,3-Butanediol | 5.0 g |
| Pure water | 42.54 g |

### Comparative Example 1

An adhesive gel sheet for living organisms of Comparative Example 1 was obtained in the same manner as Example 1 except for adding no carotenoid-containing emulsion in the production process of the adhesive gel sheet for living organisms of Example 1.

### Comparative Example 2

An adhesive gel sheet for living organisms of Comparative Example 2 was obtained in the same manner as Example 1 except for changing the composition of the hydrogel in the production process of the adhesive gel sheet for living organisms of Example 1 to the composition shown below.

| | |
|---|---|
| κ-Carrageenan | 0.20 g |
| Glucomannan (from konnyaku potato) | 0.20 g |
| Pigskin-derived acid-treated gelatin | 0.50 g |
| Magnesium chloride | 0.2 g |
| 1,3-Butanediol | 5.0 g |
| Pure water | 43.89 g |

### Comparative Example 3

An adhesive gel sheet for living organisms of Comparative Example 3 was obtained in the same manner as Example 1 except for changing the composition of the hydrogel in the production process of the adhesive gel sheet for living organisms of Example 1 to the composition shown below.

| | |
|---|---|
| κ-Carrageenan | 0.20 g |
| Glucomannan (from konnyaku potato) | 0.20 g |
| 1,3-Butanediol | 5.0 g |
| Pure water | 44.59 g |

(Evaluation of Adhesive Gel Sheet for Living Organisms)

Using the adhesive gel sheets for living organisms of Examples 1-5 and Comparative Examples 1-3 shown above, the following evaluations were performed and the evaluation results are shown in Table 1 below. In each of organoleptic evaluations for operativity, moisture retentivity, skin barrier property and skin-beautifying effect, ten monitors were each caused to use (stick on the face) the adhesive gel sheets for living organisms of Examples 1-5 and Comparative Examples 1-3 shown above, and the averages of the evaluations are shown in Table 1.

(1) Exudation Property

Each of the adhesive gel sheets for living organisms of Examples 1-5 and Comparative Examples 1-3 was cut into a size of 2 cm by 2 cm. Then, it was left at rest on a filter paper No. 2 produced by ADVANTEC Co., Ltd. and the degree of coloring of the filter paper caused by released water was evaluated visually.

A case where coloring caused by carotenoid was found within one minute was evaluated as A. A case where coloring was found within five minutes was evaluated as B. A case where slight coloring was found within ten minutes was evaluated as C. A case where visual judgment was impossible was evaluated as D.

(2) Operability

The adhesive gel sheets for living organisms of Examples 1-5 and Comparative Examples 1-3 were stuck by eyes for 15 minutes after face washing, and evaluation was conducted according to the following method and criteria.

When each of the adhesive gel sheets for living organisms was stuck on the face, a case where it did not cling to fingers and it was able to be stuck easily was evaluated as A. A case where it clung fingers and it took a long time to be stuck was evaluated as B. A case where it was readily ruptured and it was difficult to be stuck was evaluated as C.

(3) Moisture Retentivity

The adhesive gel sheets for living organisms of Examples 1-5 and Comparative Examples 1-3 were stuck to the face. Thirty minutes after peeling of the sheets, the horny layer moisture content was measured with a horny layer moisture analyzer (manufactured by Asahibiomed Co., Ltd.). A case where the horny layer moisture content increased by 10% or more by mass as an average of five monitors was evaluated as A. A case where the content increased by 2 to 10% by mass was evaluated as B. A case where the content changed by less than 2% by mass was evaluated as C.

(4) Skin Barrier Property

In order to evaluate a skin roughness improving action, the trans-epidermal water loss (TEWL) was measured by the following method.

That is, sticking for 15 minutes a day was repeated three days and the trans-epidermal water loss (TEWL) immediately after peeling of the adhesive gel sheet for living organisms in the third day was measured with a moisture loss analyzer (manufactured by Asahibiomed Co., Ltd.). A case where TEWL decreased by 5% or more by mass in comparison to before the test was evaluated as A. A case where TEWL decreased by 1 to 4% by mass was evaluated as B. A case where no change was found was evaluated as C.

(5) Skin-Beautifying Effect

Sticking for 15 minutes a day was repeated three days and impression about the appearance of the skin immediately after peeling of the adhesive gel sheet for living organisms in the third day was evaluated. A case where the skin texture was felt to be clearly regulated was evaluated as A. A case where the skin texture was felt to be slightly regulated was evaluated as B. A case where an impression that no change was found was provided was evaluated as C.

TABLE 1

| | Exudation | | Moisture | Skin barrier | Skin- |
|---|---|---|---|---|---|
| | property | Operativity | retentivity | property | beautifying effect |
| Example 1 | A | A | A | A | A |
| Example 2 | B | A | A | A | A |
| Example 3 | A | A | A | A | A |
| Example 4 | A | A | A | A | A |
| Example 5 | A | B | B | A | A |
| Comparative | D | A | B | C | C |
| example 1 | | | | | |
| Comparative | C | A | B | B | C |
| example 2 | | | | | |
| Comparative | C | A | B | C | B |
| example 3 | | | | | |

From the results in Table 1, it became clear that the adhesive gel sheets for living organisms of Examples 1-5 were excellent in the effect of improving the moisture retentivity or in the skin-beautifying effect to improve the skin texture. On the other hand, the adhesive gel sheet for living organisms comprising no carotenoid-containing emulsion (Comparative Example 1) failed to exhibit any particular skin-beautifying effect and was poor in moisture retaining effect. A product having a polyvalent inorganic salt content of more than 0.1% by mass (Comparative Example 2) and a product comprising no gelatin (Comparative Example 3) were poor in exudation property and failed to exhibit well the skin-beautifying effect even though they comprised a carotenoid-containing emulsion.

According to the invention, it is possible to provide an adhesive gel sheet for living organisms which may make carotenoid permeate the skin efficiently and to provide a sheet form cosmetic comprising the same.

That is, the invention may provide the following items <1> to <7>:

<1> An adhesive gel sheet for living organisms which comprises a hydrogel comprising a carotenoid-containing O/W emulsion, gelatin or a derivative thereof and a hydrophilic polymer; and further comprises a polyvalent inorganic salt, wherein the content of the polyvalent inorganic salt is 0.1% or less by mass of the entire mass of the hydrogel.
<2> The adhesive gel sheet for living organisms according to the item <1>, wherein the hydrophilic polymer is a polysaccharide that forms a thermoreversible gel.
<3> The adhesive gel sheet for living organisms according to the item <2>, wherein the polysaccharide is at least one species selected from the group consisting of agar, glucomannan, guar gum, locust bean gum, carrageenan, gellan gum, native gellan gum and xanthan gum.
<4> The adhesive gel sheet for living organisms according to any one item of <1> to <3>, further comprising a polyhydric alcohol compound.
<5> The adhesive gel sheet for living organisms according to any one item of <1> to <4>, wherein the volume average particle diameter of the O/W emulsion is 10 nm or more to 200 nm or less.
<6> The adhesive gel sheet for living organisms according to any one item of <1> to <5>, wherein the carotenoid is astaxanthin and/or an ester thereof
<7> A sheet form cosmetic comprising the adhesive gel sheet for living organisms according to any one item of <1> to <6>.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated.

All publications, patent applications, and technical standards mentioned in this specification are herein incorporated by reference to the same extent as if such individual publication, patent application, or technical standard was specifically and individually indicated to be incorporated by reference. It will be obvious to those having skill in the art that many changes may be made in the above-described details of the preferred embodiments of the present invention. It is intended that the scope of the invention be defined by the following claims and their equivalents.

1. An adhesive gel sheet for living organisms which comprises a hydrogel including a carotenoid-containing O/W emulsion, gelatin or a derivative thereof and a hydrophilic polymer; and optionally including a polyvalent inorganic salt, wherein the content of the polyvalent inorganic salt, when optionally included, is 0.1% or less by mass of the entire mass of the hydrogel.

2. The adhesive gel sheet for living organisms according to claim 1, wherein the hydrophilic polymer is a polysaccharide that forms a thermoreversible gel.

3. The adhesive gel sheet for living organisms according to claim 2, wherein the polysaccharide is at least one selected from the group consisting of agar, glucomannan, guar gum, locust bean gum, carrageenan, gellan gum, native gellan gum and xanthan gum.

4. The adhesive gel sheet for living organisms according to claim 1, further comprising a polyhydric alcohol compound.

5. The adhesive gel sheet for living organisms according to claim 1, wherein the volume average particle diameter of the O/W emulsion is from 10 nm to 200 nm.

6. The adhesive gel sheet for living organisms according to claim 4, wherein the volume average particle diameter of the O/W emulsion is from 10 nm to 200 nm.

7. The adhesive gel sheet for living organisms according to claim 1, wherein the carotenoid is astaxanthin and/or an ester thereof.

8. The adhesive gel sheet for living organisms according to claim 4, wherein the carotenoid is astaxanthin and/or an ester thereof.

9. The adhesive gel sheet for living organisms according to claim 6, wherein the carotenoid is astaxanthin and/or an ester thereof.

10. A sheet-form cosmetic comprising the adhesive gel sheet for living organisms according to claim 1.

11. A sheet-form cosmetic comprising the adhesive gel sheet for living organisms according to claim 4.

12. A sheet-form cosmetic comprising the adhesive gel sheet for living organisms according to claim 6.

13. A sheet-form cosmetic comprising the adhesive gel sheet for living organisms according to claim 7.

14. A sheet-form cosmetic comprising the adhesive gel sheet for living organisms according to claim 8.

15. A sheet-form cosmetic comprising the adhesive gel sheet for living organisms according to claim 9.
